(19) United States
(12) Patent Application Publication
(10) Pub. No.: US 2026/0048153 A1
(43) Pub. Date: Feb. 19, 2026

(54) IODINATED VICINAL-DIOL-BASED COMPOUNDS AND RADIOPAQUE HYDROGELS FORMED FROM SAME

(71) Applicant: Boston Scientific Scimed, Inc., Maple Grove, MN (US)

(72) Inventors: Cristian Parisi, Amherst, MA (US); Joseph Thomas Delaney, JR., Minneapolis, MN (US); Yen-Hao Hsu, Shrewsbury, MA (US); Rick William Dorn, Plymouth, MN (US)

(73) Assignee: Boston Scientific Scimed, Inc., Maple Grove, MN (US)

(21) Appl. No.: 19/300,152

(22) Filed: Aug. 14, 2025

Related U.S. Application Data
(60) Provisional application No. 63/683,530, filed on Aug. 15, 2024.

(51) Int. Cl. A61K 49/04; C07C 231/12; C07C 237/46; C08J 3/075; C08K 5/20

(52) U.S. Cl. CPC A61K 49/0457; C07C 231/12; C07C 237/46; C08J 3/075; C08K 5/20

(57) ABSTRACT

In some aspects, the present disclosure provides polyiodinated polyamino compounds that comprise a residue of a carboxylic-acid-substituted polyamino compound that is linked to a residue of a polyiodinated aromatic compound that comprises a plurality of vicinal diol groups, each having a proximal hydroxyl group and a distal hydroxyl group, wherein the polyiodinated polyamino compound is formed by a process that comprises reacting a single carboxylic acid group of the carboxylic-acid-substituted polyamino compound in an ester coupling reaction with a single hydroxyl group selected from the proximal and distal hydroxyl groups of the plurality of vicinal diol groups of the polyiodinated aromatic compound, thereby forming an ester group that links the residue of the carboxylic-acid-substituted polyamino compound to the residue of the polyiodinated aromatic compound. Other aspects of the present disclosure pertain to methods of making such polyiodinated polyamino compounds and systems employing such polyiodinated polyamino compounds.

### CROSS-REFERENCE TO RELATED APPLICATIONS

This application claims the benefit of U.S. Provisional Patent Application Ser. No. 63/683,530 filed on Aug. 15, 2024, the disclosure of which is incorporated herein by reference.

### FIELD

The present disclosure relates to iodinated compounds, to hydrogels formed from iodinated compounds, and to methods of making and using iodinated compounds and hydrogels, among other aspects. The iodinated compounds of the present disclosure are useful, for example, in forming hydrogels for various biomedical applications.

### BACKGROUND

Bioresorbable hydrogels with rapid crosslinking reaction rates in vivo, known by the trade name of SpaceOAR®, have become a prominent biomaterial and obtained clinical success in creating the space between prostate and rectum, tremendously improving patient safety during the cancer therapies. SpaceOAR® is based on a multi-arm polyethylene glycol (PEG) polymer with a polyol core functionalized with succinimidyl glutarate (SG) as reactive end groups which further react with trilysine to form crosslinks. A further improvement based on this application is that some of 8-Arm PEG branches are functionalized with 2,3,5-triiodobenzamide (TIB) groups, replacing part of the SG groups, in order to provide intrinsic radiopacity to the hydrogels themselves for CT-visibility. This hydrogel is known by the trade name of SpaceOAR Vue®. The hydrogels break down in-vivo over the course of ca. 6-9 months. The breakdown occurs primarily through the hydrolysis of the ester linkages on the glutarate groups.

While the above approach is highly effective, the entire functionalization process is complex, involving multiple steps, typically five steps, from commercially available hydroxyl-terminated 8-arm PEG to its functionalized form with two different end groups (TIB and SG groups) and resulting in a significant increase of the product cost.

There is a continuing need in the biomedical arts for additional hydrogels, for precursors of such hydrogels, for methods of making such hydrogels and precursors, for methods of using such hydrogels and precursors, and for systems for forming such hydrogels, among other needs.

### SUMMARY

In some aspects, the present disclosure provides polyiodinated polyamino compounds that comprise a residue of a carboxylic-acid-substituted polyamino compound that is linked to a residue of a polyiodinated aromatic compound that comprises a plurality of vicinal diol groups, each having a proximal hydroxyl group and a distal hydroxyl group, the proximal hydroxyl group being closer to a remainder of the polyiodinated aromatic compound than the distal hydroxyl group, wherein the polyiodinated polyamino compound is formed by a process that comprises reacting a single carboxylic acid group of the carboxylic-acid-substituted polyamino compound in an ester coupling reaction with a single hydroxyl group selected from the proximal and distal hydroxyl groups of the plurality of vicinal diol groups of the polyiodinated aromatic compound, thereby forming an ester group that links the residue of the carboxylic-acid-substituted polyamino compound to the residue of the polyiodinated aromatic compound.

In some embodiments, the single hydroxyl group is the proximal hydroxyl group of one of the plurality of vicinal diol groups.

In some embodiments, the single hydroxyl group is the distal hydroxyl group of one of the plurality of vicinal diol groups.

In some embodiments, which can be used in conjunction with the above aspects and embodiments, the polyiodinated aromatic compound comprises a plurality of vicinal dihydroxy-C₂-C₆-alkyl groups.

In some embodiments, which can be used in conjunction with the above aspects and embodiments, the polyiodinated aromatic compound comprises a plurality of 2,3-dihydroxypropyl groups and wherein the single hydroxyl group is the 2-hydroxyl group of one of the plurality of 2,3-dihydroxypropyl groups or the single hydroxyl group is the 3-hydroxyl group of one of the plurality of 2,3-dihydroxypropyl groups.

In some embodiments, which can be used in conjunction with the above aspects and embodiments, the polyiodinated aromatic compound is iodixanol.

In some embodiments, which can be used in conjunction with the above aspects and embodiments, the carboxylic-acid-substituted polyamino compound is a poly(amino acid) compound that comprises from two to ten primary-amine-containing side groups.

In some embodiments, which can be used in conjunction with the above aspects and embodiments, the carboxylic-acid-substituted polyamino compound is trilysine.

In other aspects, the present disclosure provides systems for forming hydrogels that comprise (a) a first composition that comprises a polyiodinated polyamino compound in accordance with any of the above aspects and embodiments and (b) a second composition that comprises a reactive multi-arm polymer that comprises a plurality of hydrophilic polymer arms having reactive end groups that are reactive with amino groups of the polyiodinated polyamino compound.

In some embodiments, the multi-arm polymer comprises three or more polymer arms linked to a core region, each of the polymer arms comprising a hydrophilic polymer segment and an electrophilic end group. In some of these embodiments, the electrophilic end group is a cyclic imide ester group. In some of these embodiments, each of the polymer arms comprises a hydrolysable ester group disposed between the hydrophilic polymer segment and the cyclic imide ester group.

In some embodiments, which can be used in conjunction with the above aspects and embodiments, the hydrophilic polymer segment is selected from polyalkylene oxide segments, polyester segments, polyoxazoline segments, polydioxanone segments, polypeptoid segments and polypeptide segments.

In some embodiments, which can be used in conjunction with the above aspects and embodiments, the core region comprises a polyol residue.

In some embodiments, which can be used in conjunction with the above aspects and embodiments, the system comprises a first composition that comprises the polyiodinated polyamino compound in a first container and a second composition that comprises the reactive multi-arm polymer in a second container. In some of these embodiments, the first container and the second container are independently selected from vials and syringe barrels.

In some embodiments, which can be used in conjunction with the above aspects and embodiments, the system further comprising a delivery device. In some of these embodiments, the delivery device comprises a first reservoir that contains the first composition and a second reservoir that contains the second composition, wherein during operation, the first and second compositions are dispensed from the first and second reservoirs, whereupon the first and second compositions interact and crosslink with one another to form the hydrogel.

In other aspects, the present disclosure pertains to medical hydrogels formed by reaction of the first composition and the second composition of a system in accordance with any of the above aspects and embodiments.

In other aspects, the present disclosure pertains to methods of making polyiodinated polyamino compounds, the methods comprising: (a) forming an ester linkage between a single carboxylic acid group of a carboxylic-acid-substituted polyamino compound in which amino groups are protected and a single hydroxyl group of a polyiodinated aromatic compound that comprises a plurality of vicinal diol groups each having a proximal hydroxyl group and a distal hydroxyl group, the proximal hydroxyl group being closer to a remainder of the polyiodinated aromatic compound than the distal hydroxyl group, wherein the proximal and distal hydroxyl groups of all but one of the plurality of vicinal diol groups are protected and a remaining vicinal diol group is not protected, and wherein the single hydroxyl group is selected from the proximal and distal hydroxyl groups of the remaining vicinal diol group, thereby creating a protected polyiodinated polyamino compound; and (b) deprotecting the protected polyiodinated polyamino compound.

In further aspects, an ester linkage formed between a single carboxylic acid group of a carboxylic-acid-substituted polyamino compound in which amino groups are protected and a single hydroxyl group of a polyiodinated aromatic compound that comprises a plurality of vicinal diol groups, with the polyiodinated aromatic compound having a lower degree of protection compared to that of the prior aspect.

In still other aspects, the present disclosure pertains to methods of making polyiodinated polyamino compounds, the methods comprising: (a) reacting a polyiodinated aromatic compound that comprises a plurality of vicinal diol groups in an ester coupling reaction with a molar excess of a carboxylic-acid-substituted polyamino compound in which amino groups are protected, thereby forming a product mixture that comprises (i) a polyiodinated polyamino compound comprising a single residue of the polyiodinated aromatic compound and a single residue of the carboxylic-acid-substituted polyamino compound in which the amino groups are protected and (ii) a polyiodinated polyamino compound comprising a single residue of the polyiodinated aromatic compound and two or more residues of the carboxylic-acid-substituted polyamino compound in which the amino groups are protected; (b) deprotecting the amino groups to provide a product mixture that comprises (i) a polyiodinated polyamino compound comprising the single residue of the polyiodinated aromatic compound and a single residue of the carboxylic-acid-substituted polyamino compound and (ii) a polyiodinated polyamino compound comprising the single residue of the polyiodinated aromatic compound and two or more residues of the carboxylic-acid-substituted polyamino compound; and (c) separating the polyiodinated polyamino compound comprising the single residue of the polyiodinated aromatic compound and the single residue of the carboxylic-acid-substituted polyamino compound from the polyiodinated polyamino compound comprising the single residue of the polyiodinated aromatic compound and the two or more residues of the carboxylic-acid-substituted polyamino compound.

In still other aspects, the present disclosure pertains to methods of making polyiodinated polyamino compounds, the methods comprising: (a) reacting a polyiodinated aromatic compound that comprises a plurality of vicinal diol groups in an ester coupling reaction with a molar excess of a carboxylic-acid-substituted polyamino compound in which amino groups are protected, thereby forming a product mixture that comprises (i) a polyiodinated polyamino compound comprising a single residue of the polyiodinated aromatic compound and a single residue of the carboxylic-acid-substituted polyamino compound in which the amino groups are protected and (ii) a polyiodinated polyamino compound comprising a single residue of the polyiodinated aromatic compound and two or more residues of the carboxylic-acid-substituted polyamino compound in which the amino groups are protected; (b) separating the polyiodinated polyamino compound comprising the single residue of the polyiodinated aromatic compound and the single residue of the carboxylic-acid-substituted polyamino compound in which the amino groups are protected from the polyiodinated polyamino compound comprising the single residue of the polyiodinated aromatic compound and the two or more residues of the carboxylic-acid-substituted polyamino compound in which the amino groups are protected; and (c) deprotecting the amino groups.

### BRIEF DESCRIPTION OF THE DRAWINGS

FIG. 1 schematically illustrates a method of making-Boc-protected trilysine, in accordance with an embodiment of the present disclosure.

FIG. 2 schematically illustrates a process of forming a partially protect iodixanol compound, in accordance with an embodiment of the present disclosure.

FIGS. 3A-3B schematically illustrate a method of making a polyiodinated polyamino compound in accordance with an embodiment of the present disclosure by ester coupling the tBoc-protected trilysine of FIG. 1 with the partially protect iodixanol compound of FIG. 2.

FIG. 3C schematically illustrates an alternative method of making a polyiodinated polyamino compound in accordance with an embodiment of the present disclosure by ester coupling the t-Boc-protected trilysine of FIG. 1 with unprotected iodixanol.

FIG. 4 schematically illustrates an isomer of the trilysine-iodixanol conjugate of FIG. 3B, in accordance with an embodiment of the present disclosure.

FIG. 5 schematically illustrates a method of forming a reactive polymer, in accordance with an embodiment of the present disclosure.

FIG. 6 schematically illustrates a method of forming covalent linkage, in accordance with an embodiment of the present disclosure.

FIG. 7 illustrates a delivery device, in accordance with an embodiment of the present disclosure.

FIG. 8 illustrates a delivery device, in accordance with another embodiment of the present disclosure.

### DETAILED DESCRIPTION

In various aspects, the present disclosure pertains to polyiodinated polyamino compounds (i.e., compounds that comprise a plurality of iodine groups and a plurality of amino groups) that comprise a polyamino moiety that is linked to a vicinal-diol-substituted polyiodinated aromatic moiety through an ester-containing linkage. Such polyiodinated polyamino compounds are useful, for example, as crosslinking agents for hydrogel formation.

In various embodiments, the polyiodinated polyamino compounds comprise a residue of a carboxylic-acid-substituted polyamino compound that is linked to a residue of a polyiodinated aromatic compound having plurality of vicinal diol groups, also referred to herein for convenience as a vicinal-diol-substituted polyiodinated aromatic compound. Such polyiodinated polyamino compounds may be formed by an esterification reaction in which a single carboxylic acid group of the carboxylic-acid-substituted polyamino compound is reacted with a single hydroxyl group of one of the plurality of vicinal diol groups of the vicinal-diol-substituted polyiodinated aromatic compound to form an ester-containing linkage.

In various embodiments, polyiodinated polyamino compounds of the present disclosure comprise a polyamino moiety having a plurality (two, three, four, five, six, seven, eight, nine, ten or more) amino groups. For example, the polyamino moiety may comprises a plurality (two, three, four, five, six, seven, eight, nine, ten or more) of —(CH₂)ₓ—NH₂ groups where x is 0, 1, 2 3, 4, 5 or 6. In some of these embodiments, the polyamino moiety may comprises a plurality of —(CH₂)ₓ—NH₂ groups disposed along a polymeric moiety (defined herein as a moiety comprising 2, 3, 4, 5, 6, 7, 8, 9, 10 or more monomer residues). In some embodiments, the polymeric moiety may be selected from a polyamide moiety, a polyalkylene moiety, or a polysaccharide moiety, among others.

Examples of carboxylic-acid-substituted polyamino compounds which can be used to form polyiodinated polyamino compounds in accordance with the present disclosure include poly(amino acids) that comprise a plurality of primary-amine-containing side groups (e.g., 2, 3, 4, 5, 6, 7, 8, 9, 10 or more primary-amine-containing side groups), such as, for example, poly(amino acids) that comprise lysine and/or ornithine (e.g., polylysine compounds such as dilysine, trilysine, tetralysine, pentalysine, etc., polyornithine compounds such as diornithine, triornithine, tetraornithine, pentaornithine, etc., and poly(lysine-co-ornithine) compounds), as well as carboxylic-acid-terminated polyamines such as carboxylic-acid-terminated poly(allyl amine), or carboxylic-acid-terminated poly(vinyl amine), or carboxylic-acid-terminated chitosan.

In various embodiments, the vicinal-diol-substituted polyiodinated polyamino compounds of the present disclosure comprise a vicinal-diol-substituted polyiodinated aromatic moiety comprising a plurality (e.g., two, three, four, five, six, seven, eight, nine, or more) of iodine groups and a plurality (e.g., one, two, three, four, five, six, seven, eight, nine, or more) of vicinal diol groups, examples of which include vicinal dihydroxy-C₂-C₆-alkyl groups (i.e., dihydroxy-C₂-C₆-alkyl groups in which the two hydroxyl groups are positioned on adjacent carbon atoms), among other possibilities. Because the two hydroxyl groups of each vicinal diol group are on adjacent carbon atoms, the vicinal diol group will comprise a proximal hydroxyl group and a distal hydroxyl group, with the proximal hydroxyl group being closer to a remainder of the vicinal-diol-substituted polyiodinated aromatic compound than the distal hydroxyl group.

In some embodiments, the vicinal-diol-substituted polyiodinated aromatic moiety comprises a single benzene ring with three iodine groups linked to the benzene ring and one, two or three vicinal dihydroxy-C₂-C₆-alkyl groups linked to the benzene ring. A particular example of a vicinal dihydroxy-C₂-C₆-alkyl group is a 2,3-dihydroxypropyl group, in which the proximal hydroxyl group is the 2-hydroxyl group and the distal hydroxyl group is the 3-hydroxyl group. In some embodiments, the polyiodinated aromatic moiety comprises two or more benzene rings with three iodine groups linked to each benzene ring and two or three vicinal dihydroxy-C₂-C₆-alkyl groups linked to each benzene ring. The iodine groups are typically directly attached to the benzene ring. In various embodiments, it is beneficial to have iodine in the 1, 3 and 5 positions, although other arrangements are possible. The vicinal dihydroxy-C₂-C₆-alkyl groups may be directly linked to the benzene ring or linked to the benzene ring through a linkage that contains one or more amine groups, one or more carbonyl groups, one or more amide groups, one or more ether groups, one or more alkyl groups, or a combination thereof, among others. In embodiments that comprise two or more benzene rings, the benzene rings are either directly linked each other or are linked to each other through a suitable linkage, for example, a linkage that contains one or more amine groups, one or more carbonyl groups, one or more amide groups, one or more ether groups, one or more alkyl groups, or a combination thereof, among others.

Examples of vicinal-diol-substituted polyiodinated aromatic compounds that can be used to form vicinal-diol-substituted polyiodinated aromatic moieties in accordance with the present disclosure include iodixanol,

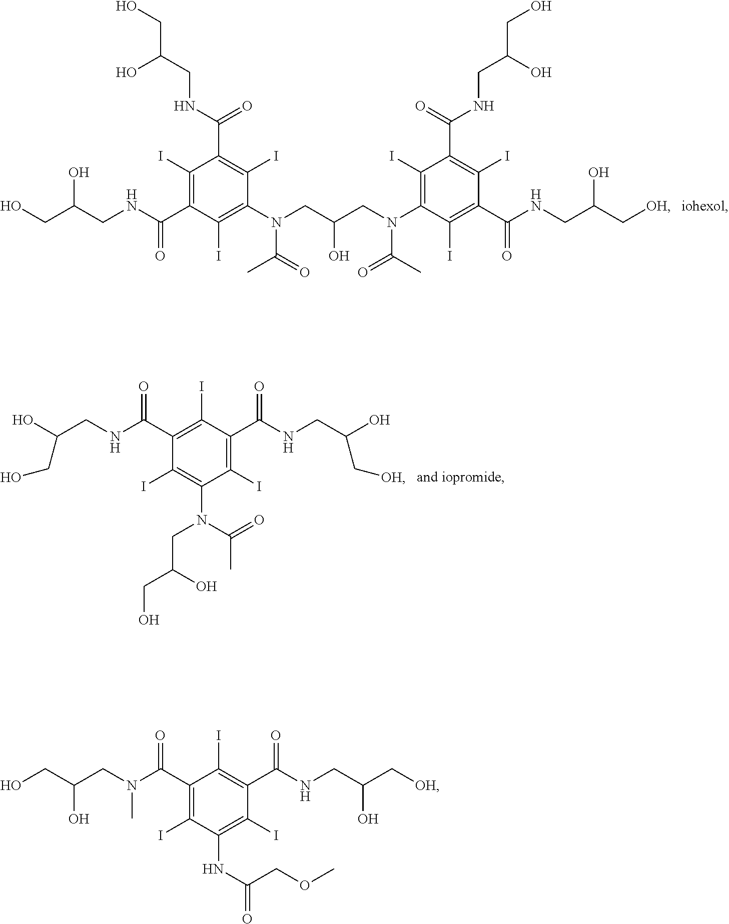 [SMILES: CC(=O)N(CC(O)CN(C(C)=O)c1c(I)c(C(=O)NCC(O)CO)c(I)c(C(=O)NCC(O)CO)c1I)c1c(I)c(C(=O)NCC(O)CO)c(I)c(C(=O)NCC(O)CO)c1I ; CC(=O)N(CC(O)CO)c1c(I)c(C(=O)NCC(O)CO)c(I)c(C(=O)NCC(O)CO)c1I ; COCC(=O)Nc1c(I)c(C(=O)NCC(O)CO)c(I)c(C(=O)N(C)CC(O)CO)c1I]

in which vicinal dihydroxy-C₂-C₆-alkyl groups are linked to a benzene group through a linkage that contains an amide group (i.e., a 2,3-dihydroxypropylaminocarbonyl group and/or an N-methyl-2,3-dihydroxypropylaminocarbonyl group) and in which benzene rings are linked together through an alkyl group, specifically, a hydroxy-substituted alkyl group, and two amide groups (i.e., through an -acetamido-2-hydroxy-propyl-acetamido-linkage).

Further examples of vicinal-diol-substituted polyiodinated aromatic compounds

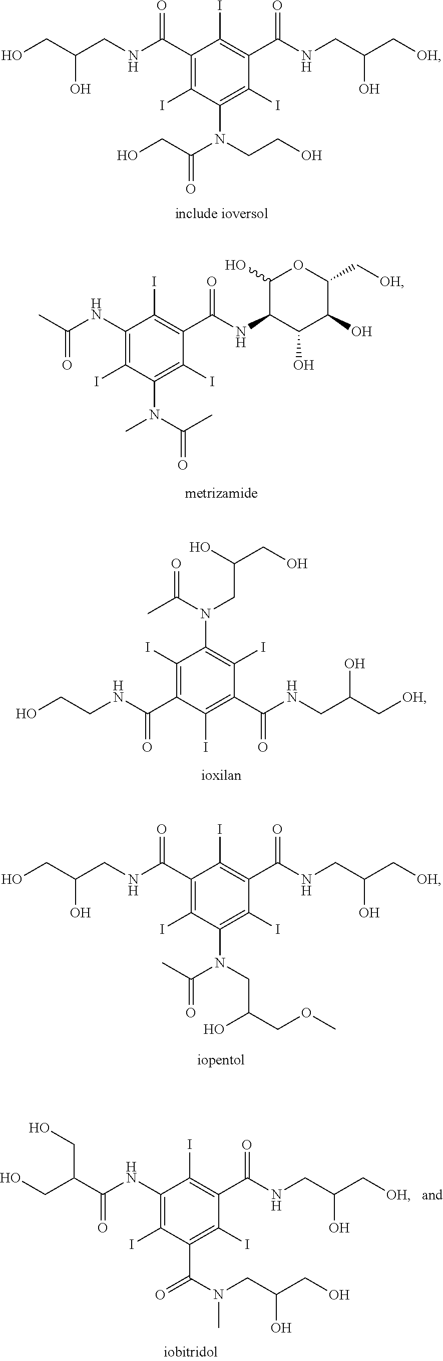 [SMILES: O=C(NCC(O)CO)c1c(I)c(C(=O)NCC(O)CO)c(I)c(N(CCO)C(=O)CO)c1I ; CC(=O)Nc1c(I)c(C(=O)N[C@H]2C(O)O[C@H](CO)[C@@H](O)[C@@H]2O)c(I)c(N(C)C(C)=O)c1I ; CC(=O)N(CC(O)CO)c1c(I)c(C(=O)NCCO)c(I)c(C(=O)NCC(O)CO)c1I ; COCC(O)CN(C(C)=O)c1c(I)c(C(=O)NCC(O)CO)c(I)c(C(=O)NCC(O)CO)c1I ; CN(CC(O)CO)C(=O)c1c(I)c(NC(=O)C(CO)CO)c(I)c(C(=O)NCC(O)CO)c1I]

As previously indicated, in some aspects, the present disclosure pertains to processes of making polyiodinated polyamino compounds by reacting a single carboxylic acid group of a carboxylic-acid-substituted polyamino compound with a single hydroxyl group of one of the plurality of vicinal diol groups of a vicinal-diol-substituted polyiodinated aromatic compound to form an ester linkage.

The ester linkage is typically formed in the presence of a suitable coupling agent, for example, a carbodiimide coupling agent such as N,N′-dicyclohexylcarbodiimide (DCC), 1-ethyl-3-(3-dimethyl′propyl)carbodiimide (EDC), 1,3-diisopropylcarbodiimide (DIC), N-hydroxybenzotriazole (HOBt), BOP reagent, TBTU, and/or another coupling agent. The ester may also be formed in the presence of a suitable base such as 4-dimethylaminopyridine (DMAP) or triethyl amine or any tertiary amines.

To prevent the amino groups of the carboxylic-acid-substituted polyamino compound from reacting with the carboxylic acid group of the carboxylic-acid-substituted polyamino compound, the amino groups of the carboxylic-acid-substituted polyamino compound are protected using a suitable protective group. Examples of protective groups for this purpose include tert-butoxycarbonyl (Boc) groups, carboxybenzyl (CBz) or (Z) groups, trifluoroacetyl (TFA) groups, and 9-fluorenylmethoxycarbonyl (Fmoc) groups, among others.

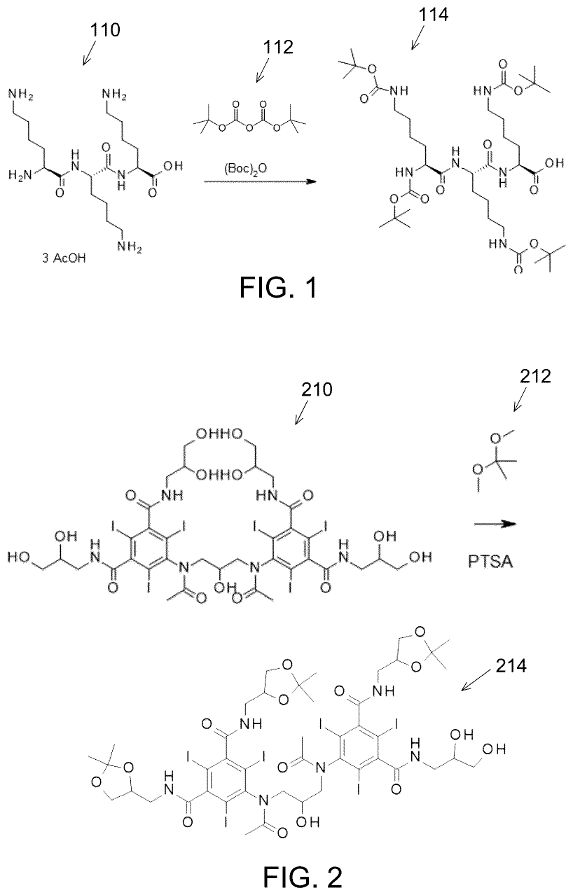

In a particular embodiments shown in FIG. 1, the amino groups of a carboxylic-acid-substituted polyamino compound, specifically trilysine (110) (the acetate form is shown), are protected using di-tert-butyl dicarbonate (112), thereby forming tBoc-protected trilysine (114). This leaves the carboxylic acid group of the tBoc-protected trilysine (114) available for ester coupling.

In various embodiments, a vicinal-diol-substituted polyiodinated aromatic compound having plurality of vicinal diol groups may be partially protected with a suitable protective agent in order protect all but one of the plurality of vicinal diol groups. For example, all but one of the plurality of vicinal diol groups may be protected with 2,2-dimethoxypropane, formaldehyde or acetaldehyde to obtain a partially-acetal-protected polyiodinated aromatic compound.

In a particular example shown in FIG. 2, a vicinal-diol-substituted polyiodinated aromatic compound having plurality of vicinal diol groups, specifically, iodixanol (210) is partially protected using 2, 2-dimethoxypropane (112) in the presence of an organic solvent such as dimethylformamide and p-toluenesulfonic acid (PTSA), to obtain partially-acetal-protected iodixanol (214) in which a single vicinal diol group remains unprotected. The partial protection is obtained by adjusting the molar ratio of the iodixanol (210) to the 2, 2-dimethoxypropane (112) (e.g., an iodixanol:2,2-dimethoxypropane ratio ranging from 1:4 to 1:3) and/or by isolating the desired partially-acetal-protected iodixanol product (e.g., using preparative-scale chromatography). The partial protection can be also obtained with an initial full protection using the same protective group, followed by a targeted partial deprotection to obtain the desired final partial deprotection.

Then, the partially-acetal-protected polyiodinated aromatic compound is reacted with the amino-protected carboxylic-acid-substituted polyamino compound in an ester coupling reaction using a suitable coupling agent to form a protected polyiodinated polyamino compound. This is followed by deprotection, to form a final polyiodinated polyamino compound.

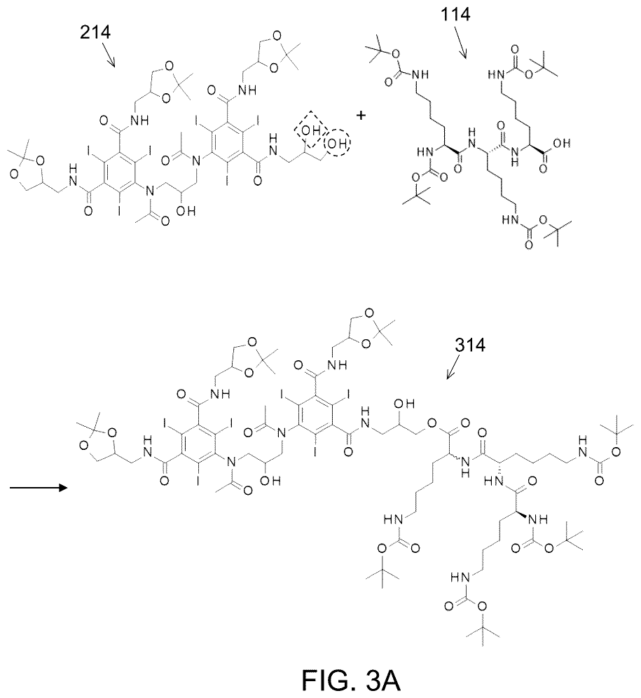

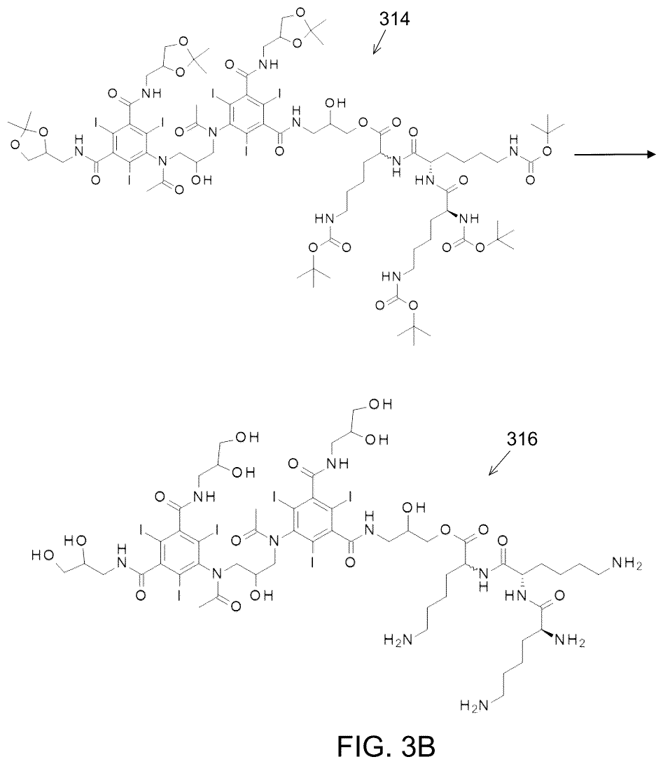

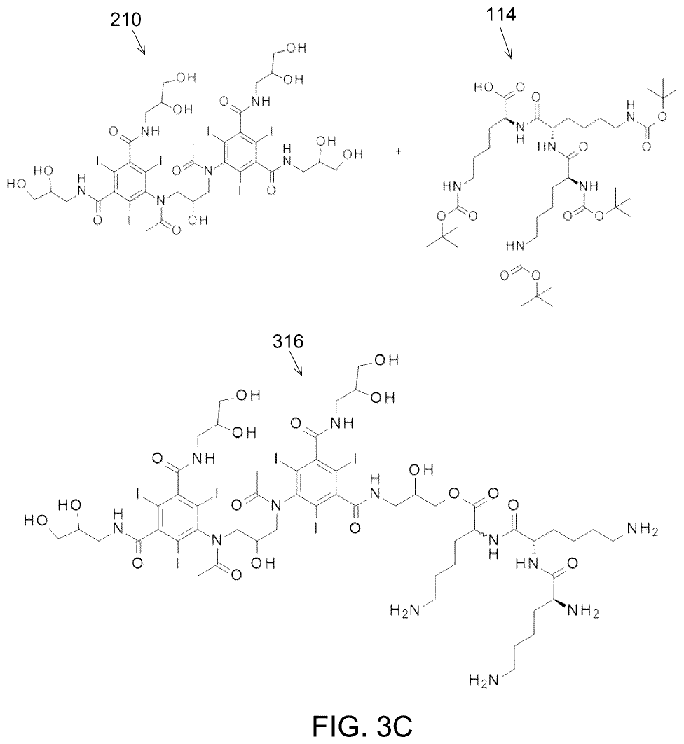

In a particular embodiment shown in FIG. 3A, the partially-acetal-protected iodixanol (214) of FIG. 2 is reacted with the tBoc-protected trilysine (114) of FIG. 1 in the presence of N,N′-dicyclohexylcarbodiimide (DCC) as a coupling agent and 4-dimethylaminopyridine (DMAP) as a catalyst in a suitable solvent to form a protected polyiodinated polyamino compound (314) that comprises a tBoc-protected trilysine residue and an acetal-protected iodixanol residue that are linked to each other through an ester group. It is noted that, in this particular case, the iodixanol has a central unprotected hydroxyl group that does not react to any significant degree with the t-Boc-protected trilysine, presumably due to steric effects.

Then as shown in FIG. 3B, the acetal protection is removed from the acetal-protected iodixanol residue and the tBoc protection is removed from the t-Boc-protected trilysine residue, for example, by exposure to an acid such as HCl or trifluoroacetic acid to provide a final polyiodinated aromatic compound (316), which comprises a trilysine residue that is linked to an iodixanol residue through an ester group.

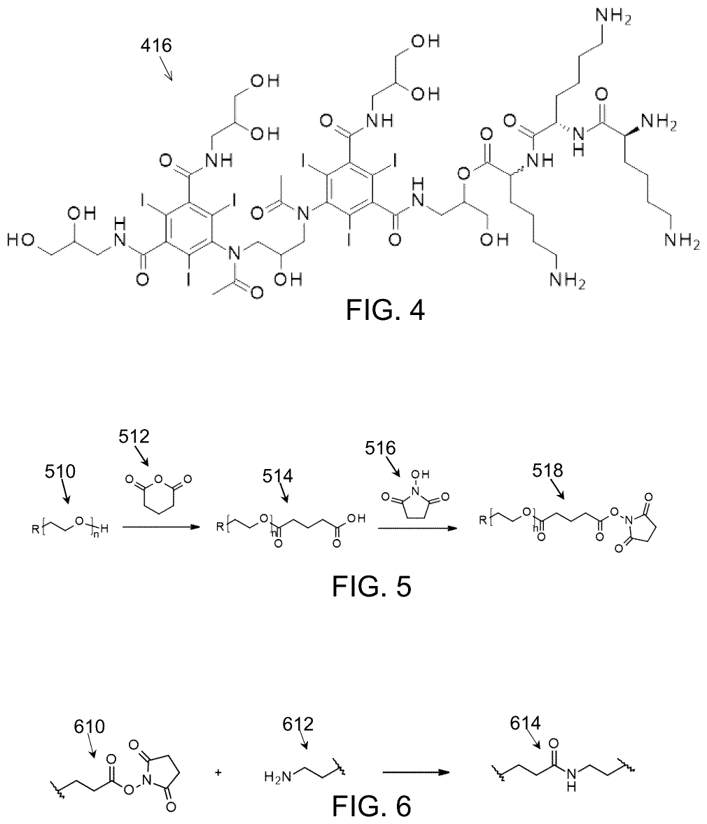

It is noted that in FIG. 3A, the distal hydroxyl group (designated with a dashed circle) of the 2,3-dihydroxypropylaminocarbonyl group of the partially-acetal-protected iodixanol (214) is linked to the tBoc-protected trilysine (114). However, in other embodiments, an isomer may be formed in which the proximal hydroxyl group (designated with a dashed diamond) is linked to the trilysine. The polyiodinated aromatic compound (416) resulting from linkage through the proximal hydroxyl group is shown in FIG. 4.

In another particular embodiment shown in FIG. 3C, unmodified iodixanol (210) is reacted with the t-Boc-protected trilysine (114) of FIG. 1 in the presence of 1-ethyl-3-(3-dimethyl′propyl)carbodiimide (EDC) as a coupling agent in a suitable solvent to form a polyiodinated polyamino compound that comprises a t-Boc-protected trilysine residue and an iodixanol residue that are linked to each other through an ester group. An excess of iodixanol is used to preferentially form iodixanol in which a single t-Boc-protected trilysine residue is attached to a single iodixanol residue (for example, an unmodified iodixanol:t-Boc-protected trilysine ratio ranging from 1:1 to 2:1 to 4:1 to 8:1 or greater). Subsequently, the t-Boc protection is removed from the t-Boc-protected trilysine residue, for example, by exposure to an acid such as HCl or trifluoroacetic acid to provide a final polyiodinated aromatic compound (316), which comprises a trilysine residue that is linked to an iodixanol residue through an ester group. Reaction products that contain an iodixanol residue and a single trilysine residue may be separated from other products such as any unreacted iodixanol and reaction products that contain an iodixanol residue and two or more trilysine residues via various purification techniques, including crystallization, precipitation, extractions and preparative-scale chromatography, among others.

In other aspects of the present disclosure, crosslinked reaction products of (a) a polyiodinated polyamino compound such as those described above and (b) a reactive multi-arm polymer that comprises a plurality of polymer arms that have reactive end groups that are reactive with the amino groups of the polyiodinated polyamino compound are provided.

In some embodiments, the crosslinked reaction products are hydrogels. As used herein, a “hydrogel,” which may also be referred to herein as a “crosslinked hydrogel,” is a crosslinked polymer that contains water or can absorb water but does not dissolve when placed in water.

In various embodiments, such crosslinked products are visible on fluoroscopy. In various embodiments, such crosslinked products have a radiopacity that is greater than 100 Hounsfield units (HU), beneficially anywhere ranging from 100 HU to 250 HU to 500 HU to 750 HU to 1000 HU to 2000 HU or more (in other words, ranging between any two of the preceding numerical values), for example, when measured on a bench-top micro CT system such as Xtreme CT from Scanco Medical (Wangen-Brüttisellen, Switzerland) or similar.

Such crosslinked products may be formed in vivo (e.g., using a delivery device like that described below), or such crosslinked products may be formed ex vivo and subsequently administered to a subject. Such crosslinked products can be used in a wide variety of biomedical applications, including medical devices, implants, and pharmaceutical compositions.

In various embodiments, the reactive end groups of the reactive multi-arm polymer and the amino groups of the polyiodinated polyamino compound react with one another via an amide coupling reaction to form a crosslinked product.

In some embodiments, reactive multi-arm polymers in accordance with the present disclosure include reactive multi-arm polymers that comprise a plurality of polymer arms linked to a core region, where the polymer arms comprise a hydrophilic polymer segment. One end of the hydrophilic polymer segment is covalently attached to the core region through a suitable linkage, and a first reactive moiety is covalently attached to an opposite end of the hydrophilic polymer segment through a suitable linkage.

Reactive multi-arm polymers in accordance with the present disclosure include polymers having from 3 to 100 arms, for example ranging anywhere from 3 to 4 to 5 to 6 to 7 to 8 to 9 to 10 to 11 to 12 to 15 to 20 to 25 to 50 to 75 to 100 arms (in other words, having a number of arms ranging between any two of the preceding values).

Reactive end groups include those that comprise electrophilic groups. Electrophilic groups may be selected, for example, from cyclic imide ester groups, such as succinimide ester groups,

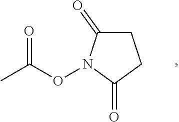

maleimide ester groups, glutarimide ester groups, diglycolimide ester groups, phthalimide ester groups, and bicyclo[2.2.1]hept-5-ene-2,3-dicarboxylic acid imide ester groups,

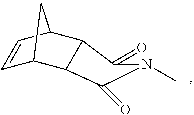

imidazole ester groups, imidazole carboxylate groups and benzotriazole ester groups, among other possibilities.

The electrophilic groups may be linked to the hydrophilic polymer segment and the hydrophilic polymer segment may be linked to the core through any suitable linking moiety, which may be selected, for example, from a bond, a linking moiety that comprises an alkyl group, a linking moiety that comprises an ether group, a linking moiety that comprises an ester group, a linking moiety that comprises an amide group, a linking moiety that comprises an amine group, a linking moiety that comprises a carbonate group, a linking moiety that comprises a urethane group, a linking moiety that comprises a urea group, a linking moiety that comprises a ketone group, or a linking moiety that comprises a combination of two or more of any of the foregoing groups, among others. In various embodiments, the linking moiety comprises a hydrolysable ester group.

Hydrophilic polymer segments can be selected from any of a variety of synthetic, natural, or hybrid synthetic-natural hydrophilic polymer segments. Examples of hydrophilic polymer segments include those that are formed from one or more hydrophilic monomers selected from the following: C₁-C₆-alkylene oxides (e.g., ethylene oxide, propylene oxide, tetramethylene oxide, etc.), polar aprotic vinyl monomers (e.g. N-vinyl pyrrolidone, acrylamide, N-methyl acrylamide, dimethyl acrylamide, N-vinyl imidazole, 4-vinylimidazole, sodium 4-vinylbenzenesulfonate, etc.), dioxanone, ester monomers (e.g. glycolide, lactide, β-propiolactone, β-butyrolactone, γ-butyrolactone, γ-valerolactone, δ-valerolactone, ε-caprolactone, etc.), oxazoline monomers (e.g., oxazoline and 2-alkyl-2-oxazolines, for instance, 2-(C₁-C₆ alkyl)-2-oxazolines, including various isomers, such as 2-methyl-2-oxazoline, 2-ethyl-2-oxazoline, 2-n-propyl-2-oxazoline, 2-isopropyl-2-oxazoline, 2-n-butyl-2-oxazoline, 2-isobutyl-2-oxazoline, 2-hexyl-2-oxazoline, etc.), 2-phenyl-2-oxazoline, N-isopropylacrylamide, amino acids, peptoids (e.g. N-substituted glycines, including N-methyl glycine or sarcosine) and sugars.

Hydrophilic polymer segments may be selected, for example, from the following polymer segments: polyether segments including poly(C₁-C₆-alkylene oxide) segments such as poly(ethylene oxide) (PEO) (also referred to as polyethylene glycol or PEG) segments, poly(propylene oxide) segments, poly(ethylene oxide-co-propylene oxide) segments, polymer segments formed from one or more polar aprotic vinyl monomers, including poly(N-vinyl pyrrolidone) segments, poly(acrylamide) segments, poly(N-methyl acrylamide) segments, poly(dimethyl acrylamide) segments, poly(N-vinylimidazole) segments, poly(4-vinylimidazole) segments, and poly(sodium 4-vinylbenzenesulfonate) segments, polydioxanone segments, polyester segments including polyglycolide segments, polylactide segments, poly(lactide-co-glycolide) segments, poly(β-propiolactone) segments, poly(β-butyrolactone) segments, poly(γ-butyrolactone) segments, poly(γ-valerolactone) segments, poly(δ-valerolactone) segments, and poly(ε-caprolactone) segments, polyoxazoline segments including poly(2-C₁-C₆-alkyl-2-oxazoline segments) such as poly(2-methyl-2-oxazoline) segments, poly(2-ethyl-2-oxazoline) segments, poly(2-propyl-2-oxazoline) segments, poly(2-isopropyl-2-oxazoline) segments, and poly(2-n-butyl-2-oxazoline) segments, poly(2-phenyl-2-oxazoline) segments, poly(N-isopropylacrylamide) segments, polypeptoid segments (e.g., poly(N-substituted glycines), including polysarcosine), polypeptide segments, and polysaccharide segments. Polysaccharide segments include those that contain one or more uronic acid species, such as galacturonic acid, glucuronic acid and/or iduronic acid, with particular examples of polysaccharide segments including alginic acid, hyaluronic acid, pectin, agaropectin, carrageenan, gellan gum, gum arabic, guar gum, xanthan gum, and carboxymethyl cellulose moieties.

Polymer segments for use in the multi-arm polymers of the present disclosure typically contain from 10 monomer units or less to 1000 monomer units or more, for example, ranging anywhere from 5 to 10 to 20 to 50 to 100 to 200 to 500 to 1000 to 2000 monomer units.

In certain embodiments, the core region comprises a residue of a polyhydroxy compound comprising three or more hydroxyl groups, also referred to herein as a “polyol”, which is used to form the polymer arms. In certain beneficial embodiments, the core region comprises a residue of a polyol that contains from 3 to 100 hydroxyl groups.

Illustrative polyols may be selected, for example, from straight-chained, branched and cyclic aliphatic polyols including straight-chained, branched and cyclic polyhydroxyalkanes, straight-chained, branched and cyclic polyhydroxy ethers, including polyhydroxy polyethers, straight-chained, branched and cyclic polyhydroxyalkyl ethers, including polyhydroxyalkyl polyethers, straight-chained, branched and cyclic sugars and sugar alcohols, such as glycerol, mannitol, sorbitol, inositol, xylitol, quebrachitol, threitol, arabitol, erythritol, pentaerythritol, dipentaerythritol, tripentaerythritol, adonitol, hexaglycerol, dulcitol, fucose, ribose, arabinose, xylose, lyxose, rhamnose, galactose, glucose, fructose, sorbose, mannose, pyranose, altrose, talose, tagatose, pyranosides, sucrose, lactose, and maltose, polymers (defined herein as two or more units) of straight-chained, branched and cyclic sugars and sugar alcohols, including oligomers (defined herein as ranging from two to ten units, including dimers, trimers, tetramers, pentamers, hexamers, heptamers, octamers, enneamers and decamers) of straight-chained, branched and cyclic sugars and sugar alcohols, including the preceding sugars and sugar alcohols, starches, amylose, dextrins, cyclodextrins, as well as polyhydroxy crown ethers, and polyhydroxyalkyl crown ethers. Illustrative polyols also include aromatic polyols including 1,1,1-tris(4′-hydroxyphenyl) alkanes, such as 1,1,1-tris(4-hydroxyphenyl)ethane, and 2,6-bis(hydroxyalkyl)cresols, among others.

Illustrative polyols also include polyhydroxylated polymers. For example, in some embodiments, the core region comprises a polyhydroxylated polymer residue such as a poly(vinyl alcohol) residue, poly(allyl alcohol), polyhydroxyethyl acrylate residue, or a polyhydroxyethyl methacrylate residue, among others. Such polyhydroxylated polymer residues may range, for example, from 3 to 100 monomer units in length.

Reactive multi-arm polymers in accordance with the present disclosure can be formed from hydroxy-terminated multi-arm polymers having arms that comprise one or more hydroxyl end groups. In some embodiments of the present disclosure, a polyol such as one of those described below, among others, may be used as multi-functional initiator for polymer chain growth. For example, the polyol may be used as an initiator for ring-opening polymerization of ethylene oxide to form polyethylene oxide (PEO) segments (also referred to a polyethylene glycol, or PEG, segments) at each of the hydroxyl groups of the polyol. The resulting hydroxyl-terminated PEG segments possess tunable hydrophilicity depending on the desired water-solubility of the resulting multi-arm polymer, for example, with increasing PEG segment length leading to increasing hydrophilicity. Hydroxyl-terminated multi-arm polymers are also available commercially. For example, hydroxyl-terminated four-arm PEG, hydroxyl-terminated six-arm PEG, and hydroxyl-terminated eight-arm PEG are available from JenKem Technology USA, Plano, TX, USA.

In some embodiments, a hydroxy-terminated multi-arm hydrophilic polymer may be reacted with a cyclic anhydride to form carboxylic-acid-terminated polymer in which carboxylic acid end groups are linked to hydrophilic polymer segments through hydrolysable ester groups. For example, terminal hydroxyl groups of the hydrophilic polymer segments may be reacted with a cyclic anhydride (e.g., glutaric anhydride, succinic anhydride, malonic anhydride, adipic anhydride, diglycolic anhydride, etc.) to form a carboxylic-acid-terminated segment such as a glutaric-acid-terminated segment, a succinic-acid-terminated segment, a malonic-acid-terminated segment, an adipic-acid-terminated segment, a diglycolic-acid-terminated segment, and so forth.

The preceding cyclic anhydrides, among others, may be reacted with a hydroxy-terminated multi-arm hydrophilic polymer under basic conditions to form a carboxylic-acid-terminated multi-arm hydrophilic polymer comprising a carboxylic acid end group that is linked to a hydrophilic polymer segment through a hydrolysable ester group. Carboxylic-acid-terminated multi-arm polymers are also available commercially. For example, carboxylic-acid-terminated four-arm PEG and carboxylic-acid-terminated eight-arm PEG (without hydrolysable ester groups) are available from JenKem Technology USA.

With reference now to FIG. 5, a cyclic anhydride, specifically glutaric anhydride 512, is reacted with a hydroxy-terminated multi-arm hydrophilic polymer, specifically a hydroxy-terminated multi-arm PEO 510, where R corresponds to a core, for example, a polyol residue core, to form a carboxylic-acid-terminated multi-arm polymer, specifically a glutaric-acid-terminated multi-arm PEO 14. (Although only one polymer arm of the multi-arm polyethylene oxide 510 is shown attached to the core R in FIGS. 5, it is to be understood that additional polymer arms are present.) In FIG. 5, n is an integer and may have a value ranging from 5 to 1000 or more. Although specific example using multi-arm PEO are illustrated, the preceding strategy is widely applicable to hydroxy-terminated polymers having hydrophilic polymer segments other PEO segments, such as the segments disclosed above.

An electrophilic moiety, such as a cyclic-imide-containing moiety, may be linked to the carboxylic-acid-terminated multi-arm hydrophilic polymer. For instance, an N-hydroxy cyclic imide compound (e.g., N-hydroxysuccinimide, N-hydroxymaleimide, N-hydroxyglutarimide, N-hydroxyphthalimide, or N-hydroxy-5-norbornene-2,3-dicarboxylic acid imide, also known as N-hydroxybicyclo[2.2.1]hept-5-ene-2,3-dicarboxylic acid imide (HONB), etc.) may be reacted with the carboxylic-acid-terminated multi-arm hydrophilic polymer in the presence of a suitable coupling agent (e.g., a carbodiimide coupling agent such as N,N′-dicyclohexylcarbodiimide (DCC), 1-ethyl-3-(3-dimethyl propyl)carbodiimide (EDC), N-hydroxybenzotriazole (HOBt), BOP reagent, and/or another coupling agent) to form an activated ester group, in particular, a cyclic imide ester group (e.g., an succinimide ester group, an maleimide ester group, an glutarimide ester group, an phthalimide ester group, a diglycolimide ester group, bicyclo[2.2.1]hept-5-ene-2,3-dicarboxylic acid imide ester group, etc.) that is linked to a hydrophilic polymer segment through a hydrolysable ester group. In this way, a number of reactive diester groups can be formed.

For example, in the particular case of N-hydroxysuccinimide as an N-hydroxy cyclic imide compound, exemplary reactive end groups include succinimidyl malonate groups, succinimidyl glutarate groups, succinimidyl succinate groups, succinimidyl adipate groups, and succinimidyl diglycolate groups, among others. In the particular case of HONB as an N-hydroxy cyclic imide compound, exemplary reactive end groups include bicyclo[2.2.1]hept-5-ene-2,3-dicarboxylic acid imidyl malonate groups, bicyclo[2.2.1]hept-5-ene-2,3-dicarboxylic acid imidyl glutarate groups, bicyclo[2.2.1]hept-5-ene-2,3-dicarboxylic acid imidyl succinate groups, bicyclo[2.2.1]hept-5-ene-2,3-dicarboxylic acid imidyl adipate groups, and bicyclo[2.2.1]hept-5-ene-2,3-dicarboxylic acid imidyl diglycolate groups, among others. In the particular case of N-hydroxymaleimide as an N-hydroxy cyclic imide compound, exemplary reactive end groups include maleimidyl malonate groups, maleimidyl glutarate groups, maleimidyl succinate groups, maleimidyl adipate groups, and maleimidyl diglycolate groups, among others. In the particular case of N-hydroxyglutarimide as an N-hydroxy cyclic imide compound, exemplary reactive end groups include glutarimidyl malonate groups, glutarimidyl glutarate groups, glutarimidyl succinate groups, glutarimidyl adipate groups, glutarimidyl diglycolate groups, among others. In the particular case of N-hydroxyphthalimide as an N-hydroxy cyclic imide compound, exemplary reactive end groups include phthalimidyl malonate groups, phthalimidyl glutarate groups, phthalimidyl succinate groups, phthalimidyl adipate groups, and phthalimidyl diglycolate groups, among others.

In a particular embodiment shown in FIG. 5, a carboxylic-acid-terminated polymer, specifically, glutaric-acid-terminated multi-arm PEO 514, is reacted with N-hydroxysuccinimide 516 in the presence of a suitable amide coupling agent, such as a carbodiimide coupling agent, to form a succinimidyl-glutarate-terminated multi-arm PEO 518. Although a specific example using multi-arm PEO is illustrated, the preceding strategy is widely applicable to multi-arm polymers having hydrophilic polymer segments other PEO segments, such as the segments disclosed above. Some succinimidyl-glutarate-terminated multi-arm polymers are also available commercially. For example, succinimidyl-glutarate-terminated four-arm PEG and succinimidyl-glutarate-terminated eight-arm PEG are available from JenKem Technology USA.

In some aspects of the present disclosure, systems are provided that are configured to deliver (a) a polyiodinated polyamino compound in accordance with the present disclosure and (b) a reactive multi-arm polymer as described herein. The polyiodinated polyamino compound and the reactive multi-arm polymer are combined under conditions such that the amino groups of the polyiodinated polyamino compound and the reactive end groups of the reactive multi-arm polymer crosslink with one another. In certain embodiments, those conditions comprise an environment having a basic pH, for example, a pH ranging from about 8.5 to about 12. Such systems can be used to form crosslinked hydrogels, either in vivo or ex vivo.

A particular example of a crosslinking reaction is illustrated in FIG. 6, which shows a covalent crosslinking reaction between a cyclic amide ester group, specifically, a succinimide ester group 610 of a reactive multi-arm polymer as described herein and an amino group 12 of a polyiodinated polyamino compound as described herein, whereby an amide linking group 614 is formed. The crosslinking reaction shown is inhibited at acidic pH, but occurs spontaneously at basic pH.

In some aspects of the present disclosure, a system is provided that comprises (a) a first composition that comprises a polyiodinated polyamino as described herein and (b) a second composition that comprises a reactive multi-arm polymer as described herein.

The first composition may be a first fluid composition comprising the polyiodinated polyamino compound or a first dry composition that comprises the polyiodinated polyamino compound, to which a suitable fluid such as water for injection, saline, etc. can be added to form a first fluid composition. In addition to the polyiodinated polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer, to which a suitable fluid such as water for injection, saline, etc. can be added to form a second fluid composition. In addition to the reactive multi-arm polymer, the second composition may further comprise additional agents, including therapeutic agents, imaging agents, colorants, tonicity adjusting agents, suspension agents, wetting agents, and pH adjusting agents as described below.

In some embodiments, the polyiodinated polyamino compound is initially combined with the reactive multi-arm polymer under conditions where crosslinking between the electrophilic moieties of the reactive multi-arm polymer and the amino groups of the polyiodinated polyamino compound is suppressed (e.g., an acidic pH, in some embodiments). Then, when crosslinking is desired, the conditions are changed such that crosslinking is increased (e.g., a change from an acidic pH to a basic pH, in some embodiments), leading to crosslinking between same, thereby forming the crosslinked product.

In particular embodiments, the system comprises (a) a first composition that comprises a polyiodinated polyamino compound as described hereinabove, (b) a second composition that comprises a reactive multi-arm polymer as described hereinabove, and (c) a third composition, specifically, an accelerant composition, that contains an accelerant that is configured to accelerate a crosslinking reaction between the polyiodinated polyamino compound and the reactive multi-arm polymer.

The first composition may be a first fluid composition comprising the polyiodinated polyamino compound that is buffered to an acidic pH or a first dry composition that comprises the polyiodinated polyamino compound and acidic buffering composition, to which a suitable fluid such as water for injection, saline, etc. can be added to form a first fluid composition comprising the polyiodinated polyamino compound that is buffered to an acidic pH. In some embodiments, for example, the acidic buffering composition may comprise monobasic sodium phosphate, among other possibilities. The first fluid composition comprising the polyiodinated polyamino compound may have a pH ranging, for example, from about 3 to about 6.5. In addition to the polyiodinated polyamino compound, the first composition may further comprise additional agents, including therapeutic agents, imaging agents, colorants, tonicity adjusting agents, suspension agents, wetting agents, and pH adjusting agents as described below.

The second composition may be a second fluid composition comprising the reactive multi-arm polymer or a second dry composition that comprises the reactive multi-arm polymer from which a fluid composition is formed, for example, by the addition of a suitable fluid such as water for injection, saline, or the first fluid composition comprising the polyiodinated polyamino compound that is buffered to an acidic pH. In addition to the reactive multi-arm polymer, the second composition may further comprise additional agents, including therapeutic agents, imaging agents, colorants, tonicity adjusting agents, suspension agents, wetting agents, and pH adjusting agents as described below.

In a particular embodiment, the first composition is a first fluid composition comprising the polyiodinated polyamino compound that is buffered to an acidic pH and the second composition comprises a dry composition that comprises the reactive multi-arm polymer. The first composition may then be mixed with the second composition to provide a prepared fluid composition that is buffered to an acidic pH and comprises the polyiodinated polyamino compound and the reactive multi-arm polymer. In a particular example, a syringe may be provided that contains the first fluid composition comprising the polyiodinated polyamino compound that is buffered to an acidic pH, and a vial may be provided that comprises the dry composition (e.g., a powder) that comprises the reactive multi-arm polymer. The syringe may then be used to inject the first fluid composition into the vial containing the reactive multi-arm polymer to form a prepared fluid composition that contains the polyiodinated polyamino compound and the reactive multi-arm polymer, which can be withdrawn back into the syringe for administration.

The third composition may be a fluid accelerant composition that is buffered to a basic pH or a dry composition that comprise a basic buffering composition to which a suitable fluid such as water for injection, saline, etc. can be added to form a fluid accelerant composition that is buffered to a basic pH. For example, the basic buffering composition may comprise sodium borate and dibasic sodium phosphate, among other possibilities. The fluid accelerant composition may have, for example, a pH ranging from about 8.5 to about 11.5. In addition to the above, the fluid accelerant composition may further comprise additional agents, including those described below.

A prepared fluid composition that is buffered to an acidic pH and comprises the polyiodinated polyamino compound and the reactive multi-arm polymer as described above (as well as additional agents in some cases), and a fluid accelerant composition that is buffered to basic pH as described above (which may include additional agents in some cases), may be combined form crosslinked hydrogels, either in vivo or ex vivo.

In some particular embodiments of the present disclosure, a kit is provided that include a first reservoir (e.g., a vial or syringe barrel) containing a first composition comprising a polyiodinated polyamino compound as described herein and an acidic buffer, a second reservoir (e.g., a vial or syringe barrel) containing a second composition comprising containing a reactive multi-arm polymer as described herein, a third reservoir (e.g., a vial or syringe barrel) containing a third composition comprising a buffered accelerant as described herein, additional apparatus, as required, for combining the first and second compositions to provide a prepared fluid composition that is buffered to an acidic pH and comprises the polyiodinated polyamino compound and the reactive multi-arm polymer, and additional apparatus for combining and delivering the prepared fluid composition and buffered accelerant to a patient.

In more particular embodiments, the kit may comprise a vial containing a reactive multi-arm polymer as described herein in dry (e.g., powdered) form, a first syringe containing a fluid composition comprising a polyiodinated polyamino compound as described herein that is buffered to an acidic pH, a second syringe containing a buffered accelerant solution as described herein, a needle and/or tube, a Y-connector, a syringe holder, a plunger cap and a vial adapter. Such components may be placed in sterile packaging, for example, in one or more packaged sterile trays.

The compositions described herein may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO2, gamma, x-ray or electron beam irradiation.

Additional agents for use in the compositions described herein include therapeutic agents, imaging agents, colorants, tonicity adjusting agents, suspension agents, wetting agents, and pH adjusting agents.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, matrix metalloproteinase inhibitors, Bcl-2 inhibitors, DNA alkylating agents, spindle poisons, poly(DP-ribose) polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxylic acid groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents (beyond the radiopaque iodine atoms that are present) such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block polymers, etc.), among others, and pH adjusting agents including various buffer solutes.

A prepared fluid composition that is buffered to an acidic pH and comprises the polyiodinated polyamino compound and the reactive multi-arm polymer as described above, and a fluid accelerant composition that is buffered to basic pH as described above, may be combined form crosslinked hydrogels, either in vivo or ex vivo.

In various embodiments, a system is provided that includes one or more delivery devices for delivering first and second compositions to a subject.

In some embodiments, the system may include a delivery device that comprises a first reservoir that contains a first composition that comprises a polyiodinated polyamino compound as described above and a second reservoir that contains a second composition that comprises a reactive multi-arm polymer that comprises a plurality of electrophilic moieties that are reactive with the amino moieties of the polyiodinated polyamino compound as described above.

In some embodiments, the system may include a delivery device that comprises a first reservoir that contains a first composition that comprises the polyiodinated polyamino compound and the reactive multi-arm polymer and is buffered to an acidic pH, such as the prepared fluid composition previously described, and a second reservoir that contains second composition, such as the fluid accelerant composition previously described.

In either case, during operation, the first composition and second composition are dispensed from the first and second reservoirs and combined, whereupon the polyiodinated polyamino compound and the reactive multi-arm polymer and crosslink with one another to form a crosslinked hydrogel.

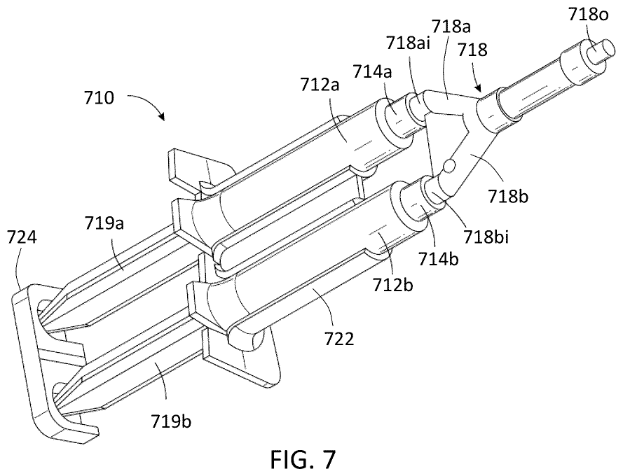

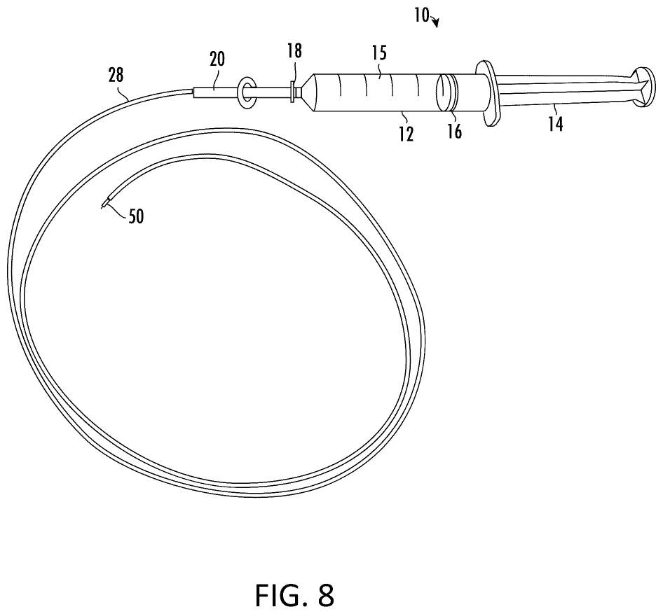

In particular embodiments, and with reference to FIG. 7, the system may include a delivery device 710 that comprises a double-barrel syringe, which includes a first barrel 712a having a first barrel outlet 714a, which first barrel contains a first fluid composition as described above, a first plunger 719a that is movable in the first barrel 712a, a second barrel 712b having a second barrel outlet 714b, which second barrel 712b contains a second fluid composition as described above, and a second plunger 719b that is movable in the second barrel 712b. In some embodiments, the device 710 may further comprise a mixing section 718 (e.g., a Y-connector) having a first mixing section inlet 718ai in fluid communication with the first barrel outlet 714a, a second mixing section inlet 718bi in fluid communication with the second barrel outlet 714b, and a mixing section outlet 7180. Also shown are a syringe holder 722 configured to hold the first and second syringe barrels 712a, 712b, in a fixed relationship and a plunger cap 724 configured to hold the first and second plungers 719a, 719b in a fixed relationship.

In some embodiments, the delivery device may further comprise a needle or catheter tube that is configured to receive the first and second fluid compositions from the first and second barrels. For example, a needle or catheter tube may be configured to form a fluid connection with an outlet of a mixing section by attaching the cannula or catheter tube to an outlet of the mixing section, for example, via a suitable fluid connector such as a Luer connector.

As another example, the catheter may be a multi-lumen catheter that comprises a first lumen and a second lumen, a proximal end of the first lumen configured to form a fluid connection with the first barrel outlet and a proximal end of the second lumen configured to form a fluid connection with the second barrel outlet. In some embodiments, the multi-lumen catheter may comprise a mixing section having a first mixing section inlet in fluid communication with a distal end of the first lumen, a second mixing section inlet in fluid communication with a distal end of the second lumen, and a mixing section outlet.

During operation, when the first and second plungers are depressed, the first and second fluid compositions are dispensed from the first and second barrels, whereupon the first and second fluid compositions mix and ultimately crosslink to form a crosslinked hydrogel, which is administered onto or into tissue of a subject. For example, the first and second fluid compositions may pass from the first and second barrels, into the mixing section via first and second mixing section inlets, whereupon the first and second fluid compositions are mixed to form an admixture, which admixture exits the mixing section via the mixing section outlet. In some embodiments, a cannula or catheter tube is attached to the mixing section outlet, allowing the admixture to be administered to a subject after passing through the cannula or catheter tube.

As another example, the first fluid composition may pass from the first barrel outlet into a first lumen of a multi-lumen catheter and the second fluid composition may pass from the second barrel outlet into a second lumen of the multi-lumen catheter. In some embodiments the first and second fluid compositions may pass from the first and second lumen into a mixing section at a distal end of the multi-lumen catheter via first and second mixing section inlets, respectively, whereupon the first and second fluid compositions are mixed in the mixing section to form an admixture, which admixture exits the mixing section via the mixing section outlet.

Regardless of the type of device that is used to mix the first and second fluid compositions or how the first and second fluid compositions are mixed, immediately after an admixture of the first and second fluid compositions is formed, the admixture is initially in a fluid state and can be administered to a subject (e.g., a mammal, particularly, a human) by a variety of techniques. Alternatively, the first and second fluid compositions may be administered to a subject independently and a fluid admixture of the first and second fluid compositions formed in or on the subject. In either approach, a fluid admixture of the first and second fluid compositions is formed and used for various medical procedures.

For example, the first and second fluid compositions or a fluid admixture thereof can be injected to provide spacing between tissues, the first and second fluid compositions or a fluid admixture thereof can be injected (e.g., in the form of blebs) to provide fiducial markers or organ marking, the first and second fluid compositions or a fluid admixture thereof can be injected for tissue augmentation or regeneration, including cosmetic tissue augmentation, the first and second fluid compositions or a fluid admixture thereof can be injected as a filler or replacement for soft tissue, the first and second fluid compositions or a fluid admixture thereof can be injected to provide mechanical support for compromised tissue, the first and second fluid compositions or a fluid admixture thereof can be injected as a scaffold, the first and second fluid compositions or a fluid admixture thereof can be injected as an embolic composition, the first and second fluid compositions or a fluid admixture thereof can be injected for seminal vesicle occlusion, the first and second fluid compositions or a fluid admixture thereof can be injected as lifting agents for internal cyst removal, and/or the first and second fluid compositions or a fluid admixture thereof can be injected as a carrier of therapeutic agents in the treatment of diseases and cancers and the repair and regeneration of tissue, among other uses. The first and second fluid compositions or a fluid admixture thereof can also be injected into a left atrial appendage during a left atrial appendage closure procedure or injected for closure of an atrial septal defect. In some embodiments, the first and second fluid compositions or a fluid admixture thereof may be injected into the left atrial appendage after the introduction of a closure device such as the Watchman® left atrial appendage closure device available from Boston Scientific Corporation.

After administration of the compositions of the present disclosure (either separately as first and second fluid compositions that mix in vivo or as a fluid admixture of the first and second fluid compositions) a crosslinked hydrogel is ultimately formed at the administration location.

During and/or after administration, the compositions of the present disclosure can be imaged using a suitable imaging technique. Typically, the imaging technique is an x-ray-based imaging technique, such as computerized tomography or X-ray fluoroscopy, or a near near-IR fluorescence spectrometry-based technique.

As seen from the above, the compositions of the present disclosure may be used in a variety of medical procedures, including the following, among others: a procedure to implant a fiducial marker comprising a crosslinked product of the first and second fluid compositions, a procedure to implant a tissue regeneration scaffold comprising a crosslinked product of the first and second fluid compositions, a procedure to implant a tissue support comprising a crosslinked product of the first and second fluid compositions, a procedure to implant a tissue bulking agent comprising a crosslinked product of the first and second fluid compositions, a procedure to implant an embolic composition comprising a crosslinked product of the first and second fluid compositions, a procedure to implant a composition comprising a crosslinked product of the first and second fluid compositions to provide seminal vessel occlusion, a procedure to implant a lifting agent comprising a crosslinked product of the first and second fluid compositions, a procedure to introduce a left atrial appendage closure composition comprising a crosslinked product of the first and second fluid compositions, a procedure to implant a therapeutic-agent-containing depot comprising a crosslinked product of the first and second fluid compositions, a tissue augmentation procedure comprising implanting a crosslinked product of the first and second fluid compositions, a procedure to introduce a crosslinked product of the first and second fluid compositions between a first tissue and a second tissue to space the first tissue from the second tissue.

The first and second fluid compositions, fluid admixtures of the first and second fluid compositions, or the crosslinked products of the first and second fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, injection for closure of an atrial septal defect, injection for seminal vessel occlusion, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Where formed ex vivo, crosslinked hydrogels may be in any desired form, including a slab, a cylinder, a coating, or a particle. In some embodiments, the crosslinked hydrogel is dried and then granulated into particles of suitable size. Granulating may be by any suitable process, for instance by grinding (including cryogrinding), homogenization, crushing, milling, pounding, or the like. Sieving or other known techniques can be used to classify and fractionate the particles. Crosslinked hydrogel particles formed using the above and other techniques may varying widely in size, for example, having an average size ranging from 50 to 950 microns.

In addition to a crosslinked hydrogel as described above, crosslinked hydrogel compositions in accordance with the present disclosure may contain additional agents, including therapeutic agents, imaging agents, colorants, tonicity adjusting agents, suspension agents, wetting agents, and pH adjusting agents as described above.

The crosslinked hydrogel compositions of the present disclosure may be sterilized using any suitable method. For example, the compositions may be autoclaved while inside a reservoir, such as a syringe barrel, vial, or ampule by heating the mixture at or to a temperature of about 121° C. Alternatively or additionally, the compositions may be sterilized via sterile filtration and/or by supercritical CO2, gamma, x-ray or electron beam irradiation.

In various embodiments, kits are provided that include one or more delivery devices for delivering the crosslinked hydrogel to a subject. Such systems may include one or more of the following: a syringe barrel, which may or may not contain a crosslinked hydrogel as described herein; a vial, which may or may not contain a crosslinked hydrogel as described here; a needle; a flexible tube (e.g., adapted to fluidly connect the needle to the syringe); and an injectable liquid such as water for injection, normal saline or phosphate buffered saline. Whether supplied in a syringe, vial, or other reservoir, the crosslinked hydrogel may be provided in dry form (e.g., powder form) or in a form that is ready for injection, such as an injectable hydrogel form (e.g., a suspension of crosslinked hydrogel particles).

FIG. 4 illustrates a syringe 10 providing a reservoir for a crosslinked hydrogel compositions as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. The syringe barrel 12 may serve as a reservoir, containing a crosslinked hydrogel composition 15 for injection through the needle 50.

The crosslinked hydrogel compositions described herein can be used for a number of purposes.

For example, crosslinked hydrogel compositions can be injected to provide spacing between tissues, crosslinked hydrogel compositions can be injected (e.g., in the form of blebs) to provide fiducial markers, crosslinked hydrogel compositions can be injected for tissue augmentation or regeneration, crosslinked hydrogel compositions can be injected as a filler or replacement for soft tissue, crosslinked hydrogel compositions can be injected to provide mechanical support for compromised tissue, crosslinked hydrogel compositions be injected as a scaffold, and/or crosslinked hydrogel compositions can be injected as a carrier of therapeutic agents in the treatment of diseases and cancers and the repair and regeneration of tissue, among other uses.

During and/or after administration, the crosslinked hydrogel compositions of the present disclosure can be imaged using a suitable imaging technique.

As seen from the above, the crosslinked hydrogel compositions of the present disclosure may be used in a variety of medical procedures, including the following, among others: a procedure to implant a fiducial marker comprising a crosslinked hydrogel, a procedure to implant a tissue regeneration scaffold comprising a crosslinked hydrogel, a procedure to implant a tissue support comprising a crosslinked hydrogel, a procedure to implant a tissue bulking agent comprising a crosslinked hydrogel, a procedure to implant a therapeutic-agent-containing depot comprising a crosslinked hydrogel, a tissue augmentation procedure comprising implanting a crosslinked hydrogel, a procedure to introduce a crosslinked hydrogel between a first tissue and a second tissue to space the first tissue from the second tissue.

The crosslinked hydrogel compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, injection for seminal vessel occlusion, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

Crosslinked hydrogel compositions in accordance with the present disclosure include lubricious compositions for medical applications, compositions for therapeutic agent release (e.g., by including one or more therapeutic agents in a matrix of the crosslinked hydrogel), and implants (which may be formed ex vivo or in vivo) (e.g., compositions for use as tissue markers, compositions that act as spacers to reduce side effects of off-target radiation therapy, cosmetic compositions, etc.).

1. A polyiodinated polyamino compound comprising a residue of a carboxylic-acid-substituted polyamino compound that is linked to a residue of a polyiodinated aromatic compound that comprises a plurality of vicinal diol groups each having a proximal hydroxyl group and a distal hydroxyl group, the proximal hydroxyl group being closer to a remainder of the polyiodinated aromatic compound than the distal hydroxyl group, wherein the polyiodinated polyamino compound is formed by a process that comprises reacting a single carboxylic acid group of the carboxylic-acid-substituted polyamino compound in an ester coupling reaction with a single hydroxyl group selected from the proximal and distal hydroxyl groups of the plurality of vicinal diol groups of the polyiodinated aromatic compound, thereby forming an ester group that links the residue of the carboxylic-acid-substituted polyamino compound to the residue of the polyiodinated aromatic compound.

2. The polyiodinated polyamino compound of claim 1, wherein the single hydroxyl group is the proximal hydroxyl group of one of the plurality of vicinal diol groups.

3. The polyiodinated polyamino compound of claim 1, wherein the single hydroxyl group is the distal hydroxyl group of one of the plurality of vicinal diol groups.

4. The polyiodinated polyamino compound of claim 1, wherein the polyiodinated aromatic compound comprises a plurality of vicinal dihydroxy-C₂-C₆-alkyl groups.

5. The polyiodinated polyamino compound of claim 1, wherein the polyiodinated aromatic compound comprises a plurality of 2,3-dihydroxypropyl groups and wherein the single hydroxyl group is the 2-hydroxyl group of one of the plurality of 2,3-dihydroxypropyl groups or the single hydroxyl group is the 3-hydroxyl group of one of the plurality of 2,3-dihydroxypropyl groups.

6. The polyiodinated polyamino compound of claim 1, wherein the polyiodinated aromatic compound is iodixanol.

7. The polyiodinated polyamino compound of claim 1, wherein the carboxylic-acid-substituted polyamino compound is a poly(amino acid) compound that comprises from two to ten primary-amine-containing side groups.

8. The polyiodinated polyamino compound of claim 1, wherein the carboxylic-acid-substituted polyamino compound is trilysine.

9. A system for forming a hydrogel that comprises (a) a first composition that comprises the polyiodinated polyamino compound of claim 1 and (b) a second composition that comprises a reactive multi-arm polymer that comprises a plurality of hydrophilic polymer arms having reactive end groups that are reactive with amino groups of the polyiodinated polyamino compound.

10. The system of claim 9, wherein the multi-arm polymer comprises three or more polymer arms linked to a core region, each of the polymer arms comprising a hydrophilic polymer segment and an electrophilic end group.

11. The system of claim 10, wherein the electrophilic end group is a cyclic imide ester group.

12. The system of claim 11, wherein each of the polymer arms comprises a hydrolysable ester group disposed between the hydrophilic polymer segment and the cyclic imide ester group.

13. The system of claim 10, wherein the hydrophilic polymer segment is selected from polyalkylene oxide segments, polyester segments, polyoxazoline segments, polydioxanone segments, polypeptoid segments, and polypeptide segments.

14. The system of claim 9, comprising a first composition that comprises the polyiodinated polyamino compound in a first container and a second composition that comprises the reactive multi-arm polymer in a second container.

15. The system of claim 14, wherein the first container and the second container are independently selected from vials and syringe barrels.

16. The system of claim 9, further comprising a delivery device.

17. The system of claim 16, wherein the delivery device comprises a first reservoir that contains the first composition and a second reservoir that contains the second composition, and wherein during operation the first and second compositions are dispensed from the first and second reservoirs, whereupon the first and second compositions interact and crosslink with one another to form the hydrogel.

18. A medical hydrogel formed by reaction of the first composition and the second composition of the system of claim 9.

19. A method of making a polyiodinated polyamino compound comprising:
forming an ester linkage between a single carboxylic acid group of a carboxylic-acid-substituted polyamino compound in which amino groups are protected and a single hydroxyl group of a polyiodinated aromatic compound that comprises a plurality of vicinal diol groups each having a proximal hydroxyl group and a distal hydroxyl group, the proximal hydroxyl group being closer to a remainder of the polyiodinated aromatic compound than the distal hydroxyl group, wherein the proximal and distal hydroxyl groups of all but one of the plurality of vicinal diol groups are protected and a remaining vicinal diol group is not protected, and wherein the single hydroxyl group is selected from the proximal and distal hydroxyl groups of the remaining vicinal diol group, thereby creating a protected polyiodinated polyamino compound; and
deprotecting the protected polyiodinated polyamino compound.

20. A method of making a polyiodinated polyamino compound comprising:
reacting a polyiodinated aromatic compound that comprises a plurality of vicinal diol groups in an ester coupling reaction with a molar excess of a carboxylic-acid-substituted polyamino compound in which amino groups are protected, thereby forming a product mixture that comprises (a) a polyiodinated polyamino compound comprising a single residue of the polyiodinated aromatic compound and a single residue of the carboxylic-acid-substituted polyamino compound in which the amino groups are protected and (b) a polyiodinated polyamino compound comprising a single residue of the polyiodinated aromatic compound and two or more residues of the carboxylic-acid-substituted polyamino compound in which the amino groups are protected;
deprotecting the amino groups to provide a product mixture that comprises (a) a polyiodinated polyamino compound comprising the single residue of the polyiodinated aromatic compound and a single residue of the carboxylic-acid-substituted polyamino compound and (b) a polyiodinated polyamino compound comprising the single residue of the polyiodinated aromatic compound and two or more residues of the carboxylic-acid-substituted polyamino compound; and
separating the polyiodinated polyamino compound comprising the single residue of the polyiodinated aromatic compound and the single residue of the carboxylic-acid-substituted polyamino compound from the polyiodinated polyamino compound comprising the single residue of the polyiodinated aromatic compound and the two or more residues of the carboxylic-acid-substituted polyamino compound.
